(19) United States
(12) Patent Application Publication
(10) Pub. No.: US 2013/0310820 A1
(43) Pub. Date: Nov. 21, 2013

(54) DEVICE AND METHOD FOR IMPROVED TREATMENT OF BODY LUMENS

(71) Applicants: Juan A. Fernandez, Buenos Aires (AR); Ramiro Pozzo, Buenos Aires (AR)

(72) Inventors: Juan A. Fernandez, Buenos Aires (AR); Ramiro Pozzo, Buenos Aires (AR)

(21) Appl. No.: 13/983,178

(22) PCT Filed: Feb. 2, 2012
(86) PCT No.: PCT/US12/23668

§ 371 Date: Aug. 1, 2013

Related U.S. Application Data
(60) Provisional application No. 61/439,511, filed on Feb. 4, 2011.

(51) Int. Cl. A61B 18/22

(52) U.S. Cl. CPC A61B 18/22
USPC 606/12

(57) ABSTRACT

A method/device are disclosed for performing procedures in body lumens using a local energy source, with quantitative information to determine treatment parameters, to accomplish efficient, safe body lumen treatment. In one embodiment, the system comprises a special optical fiber connected to a laser device capable of emitting laser radiation, receiving reflected laser radiation and processing signals as fiber is inserted into body lumen. Quantitative measurements are carried out, while advancing the optical fiber along the lumen. In another embodiment, lumen diameter and tissue reflectivity are measured along lumen's length. The system uses accumulated data to calculate/set optimal treatment parameters, such as radiation parameters and withdrawal speed, employing physical-mathematical models relating lumen characteristics to the energy/length required to efficiently treat the lumen. Once optical fiber reaches start position, lumen characteristics have been mapped along its complete length. Laser device may include a manual, automatic or semi-automatic pullback system with a sound/visual signal for guiding physician regarding optimal withdrawal speed.

### NATIONAL FILING UNDER 35 USC 371

This application is being filed as a US National stage under 35 USC 371 of PCT Application No. PCT/US12/23668, which was filed Feb. 2, 2012 and also claims the benefit of U.S. Provisional Application Ser. No. 61/240,544 filed Feb. 4, 2011, both of which are incorporated by reference herein.

### BACKGROUND OF THE INVENTION

1. Field of the Invention

The present invention relates to minimally invasive treatments of body lumens, and in particular, to the treatment of vascular disorders by using local energy-emitting devices and conveying means, based on quantitative measurements and specific models.

2. Invention Disclosure Statement

The human body comprises a number of internal lumens that perform different functions. For example, blood vessels are lumens forming part of the circulatory system, which transport blood throughout the body. There are three major types of blood vessels: the arteries, which carry the blood away from the heart, the capillaries, which enable the actual exchange of water and other substances between the blood and the tissues; and the veins, which carry blood from the capillaries back towards the heart.

Arteries and veins have the same basic structure. Three distinct layers can be identified, from inside to outside: tunica intima, tunica media and tunica adventitia. The main difference between arteries and veins is the proportions in which these components are present.

The human venous system of the lower limbs consists essentially of the superficial venous system and the deep venous system, both connected by perforating veins. The superficial system comprises the great and the small saphenous veins, while the deep venous system includes the anterior and posterior tibial veins, which converge to form the popliteal vein near the knee. The popliteal vein, in turn, becomes the femoral vein when joined by the small saphenous vein.

The venous system comprises valves, whose main function is to achieve unidirectional blood flow back to the heart. Venous valves are usually bicuspid valves, with each cusp forming a blood reservoir, which force their free surfaces together under retrograde blood pressure. As a consequence, when properly operating, retrograde blood flow is prevented, allowing only antegrade flow to the heart. A valve becomes incompetent when their cusps are unable to seal properly under retrograde pressure gradient, so retrograde blood flow occurs. When retrograde blood flow occurs, pressure increases in the lower venous sections, dilating veins and usually leading to additional valvular failure.

Valvular failure, usually referred to as venous insufficiency, is a chronic disease that can lead to skin discoloration, varicose veins, pain, swelling and ulcerations. Varicose veins refer to blood vessels that have become enlarged and twisted and have progressively lost their wall elasticity. Due to the widening of the blood vessels, vein valves cannot close completely and veins lose their ability to carry blood back to the heart. This leads to an accumulation of blood inside the vessels, enlarging and twisting the veins even more. Furthermore, varicose veins usually have a blue or purple color and may protrude twisted above the surface of the skin, this being responsible for their characteristically unattractive appearance. They are commonly formed in the superficial veins of the legs, which are subject to high pressure when standing. Other types of varicose veins include venous lakes, reticular veins and telangiectasias. A special case of venous insufficiency may occur in arterio-venous fistulae: a connection between an artery and a vein. These can be congenital, surgically created or acquired due to pathologic process. A fistula may be surgically created for hemodialysis treatments (vascular access). Arteriovenous fistula for vascular access is carried out previous to hemodialysis treatment, in order to render a vein to grow larger and stronger for easy access to the blood system. As a consequence, an adequate vascular access is achieved, through which blood is withdrawn, purified, and returned to the body. Vascular accesses are thus entranceways into the bloodstream that lie completely beneath the skin. The access is usually performed in the arm, but sometimes in the leg, and can be done by direct artery-vein anastomosis or by means of an artificial graft. Except for specific cases artery-vein anastomosis are preferred, to eliminate risks associated with insertion of foreign bodies. It is well known that vascular accesses have a high incidence of complications, finally resulting in vascular access failure. These complications can be divided into non-thrombotic and thrombotic. Regarding non-thrombotic complications, venous hypertension is one of the most important since it may cause valvular incompetence or central venous stenosis. This may lead to severe upper limb edema, skin discoloration, access dysfunction and peripheral ischemia with fingertip ulceration.

There are a number of treatments available intending to cure these kinds of vascular pathologies. Some of them consist only in relief of symptoms but they do not treat varicose veins nor prevent them from forming. These include elevating the legs by lying down or using a footstool when sitting, using elastic stockings and doing exercise.

Varicose veins are frequently treated by eliminating the insufficient veins. This forces the blood to flow through the remaining healthy veins. Various methods can be used to eliminate the problem of insufficient veins, including, sclerotherapy, surgery (vein stripping), electro-cautery, and laser treatments.

Laser treatments are usually preferred by those skilled in the art. Minimally invasive laser surgery has been improved due to new diode laser systems. In endovascular laser surgery, laser radiation applies thermal energy to the vein by means of an optical fiber, and while fiber is withdrawn, the vein closes. Ideally, closed vein eventually disappears through resorption. In these and other eases, endovascular laser treatment provides an effective technique for eliminating or diminishing skin and vascular problems.

A well known prior art describing endovascular laser ablation procedure includes a guide wire, an introducer sheath (through which optical fiber is inserted) and tumescent anesthesia (to reduce vein diameter and protect perivenous tissue).

Recently, several improvements in endovenous laser ablation devices and techniques have been developed. U.S. Patent Publication 2009/0240242 A1 by Neuberger, discloses substantial improvements in optical fiber, including a rounded tip configuration and emission of radiation radially with respect to its main axis (radial fiber). As a consequence, previously needed guide wire and tumescent anesthesia would be no longer required when carrying out endovascular laser treatment. The need of tumescent anesthesia is further reduced by the use of wavelengths that are highly absorbed in the components surrounding fiber tip, for example, 1470 nm. As a consequence, thermal effect caused in tissue by laser radiation is restricted to the zone of interest, thus preventing from damage non-target tissue (e.g., nerves are not affected by thermal effects, thus rendering a safe and virtually painless procedure).

One of the most important factors to consider when performing laser treatments in vessels is the amount of energy (or energy density) that is deposited on the vessel wall that is being treated. Non optimal energy deposition onto vessel wall may lead to inefficient treatments and/or post-surgery complications. In the first case, vein closure may be incomplete thus leading to recurrent varicose veins. In the second case, vein wall perforations can arise in those regions of high energy deposition, thus causing hemorrhages and other complications. Moreover, perivenous tissue damage is more likely to occur when uncontrolled irradiation is applied. As a consequence, nerves may be damaged and patient's recovery delayed.

According to this, laser treatment in body lumens can be improved, usually, by controlling energy deposition on tissue. Energy deposition can be controlled according to treatment needs, that is, based on quantitative measurements of the tissue to be treated, e.g. lumen diameter, tissue composition, blood flow. As a consequence, treatment safety, efficiency and velocity will be substantially enhanced.

The most common parameter that is measured is the lumen diameter, since it will usually determine energy density needed to perform treatment. Some approaches have been proposed in order to address this. For example, in U.S. Patent Publication 2009/0178289A1, Sakai et al. disclose a system and method for mechanically measuring a cavity's internal diameter using a specially designed catheter. Basically, a cavity's internal diameter is measured by inflating a balloon until contacting cavity's internal wall. A wire is attached to balloon through a mechanism that transduces balloon expansion (internal diameter) into a longitudinal displacement. Finally, this longitudinal displacement is confirmed in a measuring portion, yielding an internal diameter value. As can be inferred, this approach presents numerous disadvantages. First, since balloon needs to be inflated and deflated at every measurement site, procedure time can be undesirably long when mapping a lumen completely. Second, since direct contact is needed to perform internal diameter measurement, tissue damage on lumen's wall may occur. In addition, lumen's diameter can be modified by the process of measurement itself due to the exerted pressure, in particular in highly compliant lumens. Finally, another source of error would be the fact that a balloon's diameter is measured instead of the lumen's internal diameter.

In another variant, U.S. Patent Publication 2002/0077568A1 by Haddock, discloses the measurement of vessel length and diameter by means of an accelerometer, for atherosclerotic plaque and stent assessment. As the catheter is advanced and then pulled back, it moves (by active or passive means) and strikes the walls of the vessel, recording acceleration peaks. Acceleration peak magnitude also allows for wall stiffness estimation. This invention presents several disadvantages. Acceleration measurements must be recorded for performing integration, so a real time approach is not possible. In addition, back and forth movements are needed, thus increasing procedural time. Furthermore, vessel walls are stricken to carry out measurement, thus leading to all the subsequent drawbacks. Finally, this catheter was conceived for performing measurements only. As a consequence, physician must first perform measurement with this catheter, then withdraw it and finally a treatment element is used, making it a long and complex procedure.

In addition to previously mentioned specific disadvantages, these approaches are only intended for diagnostic purposes, i.e. they are not proposed in combination with treatment. Thus, despite the fact that these approaches would help to decide in favor of a treatment over another based on diagnostic information, treatment parameters are not directly set by means of quantitative measurements of the tissue to treat. Therefore, parameter optimization is not accomplished.

In an attempt to overcome these drawbacks, Wallace et al. disclose in U.S. Patent Publication No. 2008/0319350A1 a method and device for normalizing ablation energy (in particular, RF energy) applied to body lumens. In order to accomplish this, an expandable element with a variable resistance/inductance mounted on a special catheter measures a lumen's circumference and this information is used to determine the energy that will be delivered to tissue. The operative element is expanded by means of an inflatable balloon within it, exerting pressure on the lumen. This approach presents numerous disadvantages. As can be seen from the description, direct contact is needed between operative element and lumen's inner surface in order to measure lumen's circumference. As a consequence, lumen's diameter may be modified by the process of measurement itself, thus yielding an incorrect result, due to the exerted pressure. For instance in veins, due to their high compliance, operative element expansion may modify vein's real diameter and circumference would be overestimated. In addition, catheter disclosed is complex, as it comprises an operative element with sensing elements, related circuitry, and an inflatable balloon. Furthermore, according to the method of measurement disclosed, it would take a long procedure time to perform various measurements along lumen's length, as balloon must be inflated and deflated as many times as the number of measurement sites.

According to the previously mentioned, improvements should be carried out with respect to current treatments in order to enhance outcomes and render an easier, more efficient and reliable procedure.

Light (electromagnetic radiation) has been used to carry out distance measurements in diverse areas of application. Distance measurement can be performed using different methods, e.g. laser triangulation, autofocusing and frequency-modulated heterodyne interferometry. Laser triangulation usually presents a poor resolution in distance measurement. In addition, this method needs for separated paths for target and return beams, thus excluding it from measuring internal diameters. Autofocusing method has a better resolution, but its outcome highly depends on surface reflectivity. Moreover, since it uses a lens system, it is usually not possible to use this method for measuring internal diameters.

Frequency-modulated heterodyne interferometry presents a good resolution, measurements can be carried out rapidly and variations in surface reflectivity can be sensed. For example, in U.S. Pat. No. 5,781,297, Castore discloses a fiber optic heterodyne interferometer for distance measurement in machined components. To accomplish this, a reference and a target beam interfere at a detector producing a beat wave, whose frequency is linearly related to the distance of the target from the sensor.

There is thus a need for a minimally invasive lumen treatment that improves on the state of the art, providing an accurate, precise, predictable and reproducible outcome, based on quantitative measurements and specific models, to optimally adjust treatment parameters. The present invention addresses these needs.

### OBJECTIVES AND BRIEF SUMMARY OF THE INVENTION

It is an objective of the present invention to provide a device and method for improved treatment of body lumen disorders.

It is another objective of the present invention to treat dysfunctional vessels accurately and precisely, by using a localized, directed energy source and conveying means.

It is also an objective of the present invention to provide a device and method for safer, easier, faster, and more reliable vascular treatment by means of measuring and processing quantitative feedback parameters.

It is yet another objective of the present invention to provide a device and method for facilitating a medical procedure by automatically setting treatment parameters based on quantitative measurements and physical-mathematical models.

Briefly stated, a method/device are disclosed for performing procedures in body lumens using a local energy source, with quantitative information to determine treatment parameters, to accomplish efficient, safe body lumen treatment. In one embodiment, the system comprises a special optical fiber connected to a laser device capable of emitting laser radiation, receiving reflected laser radiation and processing signals as fiber is inserted into body lumen. Quantitative measurements are carried out, while advancing the optical fiber along the lumen. In another embodiment, lumen diameter and tissue reflectivity are measured along lumen's length. The system uses accumulated data to calculate/set optimal treatment parameters, such as radiation parameters and withdrawal speed, employing physical-mathematical models relating lumen characteristics to the energy/length required to efficiently treat the lumen. Once optical fiber reaches start position, lumen characteristics have been mapped along its complete length. Laser device may include a manual, automatic or semi-automatic pullback system with a sound/visual signal for guiding physician regarding optimal withdrawal speed. Pullback system allows device to determine fiber position inside the lumen, and automatically adjust treatment parameters according to previous mapping.

The above and other objects, features and advantages of the present invention will become apparent from the following description read in conjunction with the accompanying drawings (in which like reference numbers in different drawings designate the same elements).

### BRIEF DESCRIPTION OF FIGURES

FIG. 1 depicts a preferred embodiment of present invention describing device's main components.

FIG. 2 shows main steps of a preferred method of the present invention.

FIG. 3 depicts a preferred embodiment in which vessel features are mapped while inserting optical fiber.

### DETAILED DESCRIPTION OF PREFERRED EMBODIMENTS

As previously mentioned, laser treatment in body lumens can be usually improved by controlling energy deposition on tissue. Energy deposition can be controlled based on quantitative measurements of the tissue to be treated. The most common parameter that is measured is the lumen diameter. Prior art approaches to address this need present numerous disadvantages. In general, since these approaches carry out diameter measurement by direct contact, tissue damage on the lumen's wall may occur and/or the lumen's diameter can be highly modified by the process of measurement itself. In addition, catheters disclosed are complex. Finally, procedure time when using these approaches can be undesirably long if mapping a lumen completely.

Light can been used to carry out distance measurements. In particular, frequency-modulated heterodyne interferometry is an appropriate method since it presents a good resolution, good procedural speed and surface reflectivity variation sensitivity. It basically consists in interfering a reference and a target beam at a detector, thus producing a beat wave, whose frequency is linearly related to the distance of the target from the sensor. Furthermore, reflected beam amplitude depends on tissue reflectivity.

As a consequence, frequency-modulated heterodyne interferometry can be used for accomplishing at least two purposes when used in body lumens: first, a lumen's internal diameter can be determined; second, superficial tissue composition can be inferred by means of an appropriate model, since tissue surface reflectivity variations can be sensed. Diameter and tissue reflectivity determinations would then be used for setting treatment parameters in an optimum manner, using different models, e.g. physical, mathematical or experimental. Therefore, treatment safety, efficiency and velocity are substantially enhanced.

Although the subsequent description will concentrate on vascular applications, it will be apparent to those skilled in the art that device and method disclosed herein are applicable to other body lumens and volumes.

In general terms, the present invention discloses a system and method which generate and process quantitative measurements, thus allowing for automatically setting of treatment parameters based on appropriate models, for instance, physical-mathematical models. Measurement of vessel wall distance from optical fiber is proposed, preferably using a laser heterodyne interferometer (triangulation techniques would not be convenient due to difference in locations of emitter and receptor). Also tissue reflectivity measurement is proposed, through which some tissue properties can be inferred. Both these measurements are performed by using special optical fibers, having a lens and mirror assembly at its distal end, thus allowing for emission and reception of laser radiation. Optical fiber diameter is small enough to carry out endoluminal approaches. Laser radiation must be emitted perpendicularly to fiber's main axis (e.g. by means of a modified side-emitting fiber). Optical fiber is connected to a laser device capable of: emitting laser radiation, receiving reflected laser radiation and processing signals.

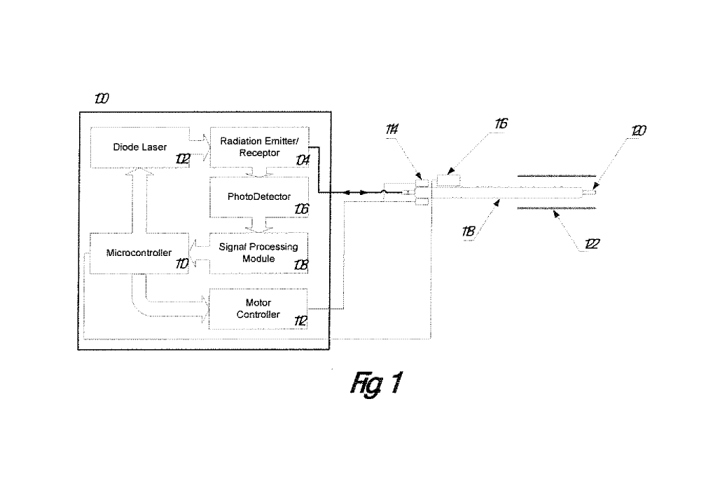

In a preferred embodiment, depicted in FIG. 1, laser device 100 comprises diode laser source 102, radiation emitter/receptor 104, photodetector 106, signal processing module 108, microcontroller 110 and motor controller 112. Laser radiation is produced in diode laser source 102, following a specific pulse pattern (e.g. sawtooth or sine). Laser radiation is coupled by optical means to radiation emitter/receptor 104, and from there it is delivered through optical fiber 120. Radiation is emitted at fiber's tip perpendicularly to its main axis, being partially reflected in vessel wall 122 and received by lens assembly (not shown), then travelling in the opposite direction through the same path as the impinging beam. In radiation emitter/receptor 104, reflected radiation is combined with a reference beam. Combined light interferes at photodetector 106, producing a beat signal, whose frequency is directly proportional to the distance from optical fiber 120 to vein wall 122. Beat signal is processed. i.e. filtered, amplified, etc, in signal processing module 108 and then fed to microcontroller 110, which is programmed to calculate vessel diameter. In addition, superficial tissue composition (e.g., calcium plaques, normal endothelium, etc) is inferred by means of its reflectivity, which is determined through reflected signal amplitude.

Using this information (vessel wall distance and probable composition) and based on a physical-mathematical analysis, also programmed in microcontroller 110, laser treatment parameters (wavelength, power, wave pattern, power density, pullback speed) are set. Laser power is set in diode laser source 102 directly by means of microcontroller 110. After pullback speed is defined, microcontroller 110 sets motor controller 112, whose main function is to drive motor 114, which performs the pullback movement of catheter 118 (previously locked with optical fiber 120 ) at the predefined velocity. Pullback speed is controlled by means of velocity sensor 116, which closes the loop by feeding a control signal to microcontroller 110.

In a preferred embodiment, a screen displays rate of removal. Additionally, a warning light and sound lets physician know when J²/cm drops too low or rises too high because withdrawal rate is too fast or too slow, respectively

Vessel wall distance from fiber's center axis and tissue reflectivity can be measured at any time in order to determine vessel diameter and composition. Once vessel diameter is determined, the system uses this information for setting treatment parameters, such as, power, energy per unit length and optimal withdrawal speed.

### EXAMPLE I

According to a mathematical analysis using 1470 nm and radial emitting fiber (Fernandez J A, Pozzo R. “Physical and Mathematical Analysis of Endovascular Laser Treatment”. Journal of Buenos Aires Phlebology and Linfology Society (SFLB), Year 3, No. 9, September-December 2008, 494-507) the time required to reach a temperature sufficient to close vein effectively (collagen denaturation temperature) can be calculated according to the following formula:

$${t}_{H} = Δ T \frac{ρ A {c}_{e}}{{P}_{LENGTH} - ( {P}_{{LENGTH}_{LOSS 1}} + {P}_{{LENGTH}_{LOSS 2}} )}$$

Where:
1. P_{LENGTH} is the power per unit of length.
2. P_{LENGTHLOSS1} is the power loss per unit of length from the intima to the adventitia [W/mm] and can be calculated as:

$${P}_{{LENGTH}_{LOSS 1}} = \frac{{k}_{v} 2 πΔ T}{ln ( {R}_{adv} / {R}_{int} )}$$

where,

kᵥ is the thermal conductivity of vessel [W/(mm ° K)]

ΔT is the temperature gradient [° K]

R_{adv} is the adventitia radius [mm]

Rᵢₙₜ is the intima radius [mm]
3. P_{LENGTHLOSS2} is the power loss per unit of length from the adventitia to the perivenous tissue [W/mm] and can be calculated as:

$${P}_{{LENGTH}_{LOSS 2}} = \frac{{k}_{t} 2 πΔ T}{ln ( {R}_{per} / {R}_{adv} )}$$

where,

kₜ is the thermal conductivity of perivenous tissue [W/(mm ° K)]

ΔT is the temperature gradient [° K]

Rₚₑᵣ is the perivenous tissue radius [mm]

R_{adv} is the adventitia radius [mm
4. ΔT, is the temperature gradient [° K]
5. density ρ and specific heat cₑ values used to calculate the time needed to increase temperature to a determined value will be:

$$ρ = \frac{1}{1000} [ \frac{g}{m {m}^{3}} ]$$

$${c}_{e} = 4.186 [ \frac{l}{{g}^{°} C .} ]$$

$${R}_{f t b } = 1 [ mm ]$$

6. Fiber diameter at emission section R_{fib} is 1 [mm]. Considering radial emitting fiber configuration, a common value of power per unit of length P_{LENGTH} is 5 [w/mm].

So, for example, for a vein of 5 mm diameter, the necessary time to reach collagen denaturation temperature will be (in a 1 mm length):

$$\begin{matrix}{t}_{H 5 m m} = Δ T \frac{ρ A {c}_{e}}{{P}_{LENGTH} - ( {P}_{{LENGTH}_{LOSS 2}} - {P}_{{LENGTH}_{LOSS 2}} )} \\ = ( 65 - 37 ) \frac{1 / 1000 π ( 6.25 - 0.25 ) 4.196}{5 - ( 0.49 - 0.02 )} \\ = 0.5 \vert s \vert\end{matrix} \qquad$$

In other words, the time required for treating 1 cm of a 5 mm diameter vein, considering heat loss, will be 5 s. As a consequence, energy per length necessary for treating a 5 mm diameter vein will be 25 J/cm. Furthermore, fiber withdrawal speed should be programmed at 1 cm/5 s=0.2 cm/s or 2 mm/s, when radiating in continuous mode.

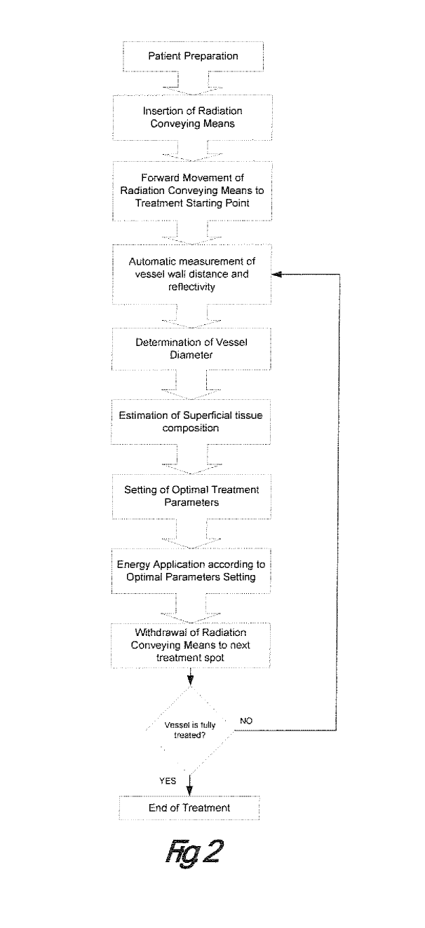

In a preferred embodiment, the treatment is performed according to the steps depicted in FIG. 2. After patient is prepared, optical fiber is inserted inside the vessel to be treated, following the introducing step of any well known endovascular laser procedure. Once optical fiber is centered and placed in the desired position, laser device is activated. When this occurs, system automatically measures vessel wall distance from the optical fiber. Accurate diameter measurements can be accomplished by at least two different methods: centering optical fiber as accurately as possible; or performing a radial measurement and then calculating the mean distance value. Since a precise measurement is required, light dispersion in the medium between optical fiber and vessel wall (e.g. blood) must be avoided or minimized. In order to accomplish this, three actions can be performed: i) using a wavelength “transparent”to the medium but adequately reflected by vessel wall, for example, approximately 700 nm for blood; ii) exsanguination; iii) injecting saline solution or other fluid capable of “displacing” the medium around fiber tip, and then using an appropriate wavelength (not absorbed by the injected fluid but reflected by vessel wall). After fiber-vessel wall distance measurement, vessel diameter is easily calculated. In addition, tissue reflectivity can be measured by means of the reflected beam amplitude. Therefore, some tissue characteristics can be inferred, for example, superficial composition of tissue, amount of deposited calcium, etc. After performing both these measurements, laser treatment parameters are set according to a physical-mathematical analysis describing the relationship between vessel diameter and composition with the energy per length required to treat the vessel.

Laser device can include an automatic pullback system or a semi-automatic system with an audible and/or visual signal for guiding physician regarding optimal withdrawal speed during manual withdrawal. As a consequence, once the physician has placed the optical fiber in the desired location, the system allows for an automatic and precise procedure, based on quantitative information to set laser treatment parameters.

In order to speed up the procedure, physician can select how frequently distance and reflectivity are measured, according the vessel characteristics. For example, when treating saphenous vein, its length could be divided in three segments, from the sapheno-femoral junction to the place where puncture is made. Thus, after activating laser device, vein diameter and its superficial composition are determined and laser treatment parameters are set. After covering the first segment (determined by the distance the optical fiber was withdrawn), vein diameter and superficial composition are determined again and laser treatment parameters are set with new values. According to this approach, treatment time is practically not affected by vein wall distance measurement. Nevertheless, measurement time can be short enough (10 ms or even less), so the distance can be measured almost continuously and measurement frequency would not be an issue. In this case, measurements can be practically made in real time before irradiation, which could be made at a continuous rate. Real time measurement would also offer greater accuracy to calculations, since they are carried out almost continuously.

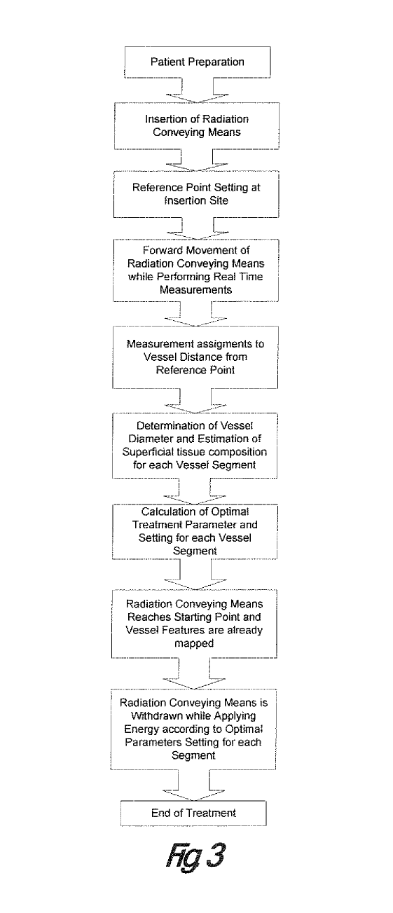

In another preferred embodiment shown in FIG. 3, a mapping of the vessel diameter and superficial composition is performed along its different segments while inserting optical fiber. Correlation between vessel characteristics and respective vessel distance from the punction site can be done by setting a reference point from which treatment will be carried out (i.e. just inside of puncture site is considered as the zero reference point). Once a reference point is chosen, optical fiber is advanced inside the vessel (as usual in endoluminal procedures) to the starting point of treatment, while performing measurements in real time along vessel length. In cases where fiber is introduced through a catheter, a sheath that is transparent to radiation must be used. Each of these measurements is then assigned to a vessel distance from the reference point. This can be accomplished by means of a system similar to that disclosed in U.S. Patent Publication 2006/0217692 A1 by Neuberger, which is able to measure fiber's speed of withdrawal. Therefore, fiber's distance from a reference point can be determined by time integration, of measured speed. Then, distance from the reference point for each vessel segment is input along with its features (vessel wall distance and reflectivity) in the controlling system, which will calculate and store optimal treatment parameters for every vessel segment. As a consequence, once optical fiber reaches treatment starting point (e.g., sapheno-femoral junction when treating great saphenous vein), vessel characteristics mapping is already complete. When the treatment begins, system retrieves optimal calculated parameters stored for the segment that is being treated. The system determines which specific segment is being treated by means of the same speed measurement device used for mapping the lumen.

Another parameter that can be taken into consideration is vessel ramifications. This is important for instance when treatment of insufficient veins also includes preventing recanalization. In another preferred embodiment, the system detects the position of vessel ramifications along the vessel to be treated and sets a higher power to apply there in order to assure a correct effect on vessel wall. As a consequence, in this case, vessel wall distance and reflectivity as well as the position of its ramifications are calculated for different segments. This is the input data for the system, which then, automatically sets optimal laser treatment parameters by means of a physical-mathematical or experimental model describing the relationship between vessel diameter, composition and ramifications with the energy per length required to treat the vessel.

In another preferred embodiment, vessel diameter can be measured using an ultrasound transducer incorporated in the fiber/catheter itself. Also, based on vessel echogenicity, superficial composition can be inferred. Once these measurements are performed, the system determines optimal treatment parameters as described previously. In another embodiment, distances and tissue composition can be obtained by different imaging technologies, such as Magnetic Resonance, Computerized Axial Tomography, or a high resolution endovascular imaging means such as Optical Coherence Tomography.

In some special clinical cases, experienced physicians may prefer to set their own treatment values considering other clinical criteria, regardless of system's automatic calculations. In spite of setting laser treatment parameters automatically after vessel characteristic measurements, the system allows the physician to adjust and store different values for diverse cases. In these cases, the system will adapt its algorithm to different regional patient features, physician preferences or to new criteria described by new experience as useful for determining treatment parameters. These can be done for example by means of artificial neural networks (ANN).

As can be seen from previous description, the system and method disclosed in the present invention have numerous advantages. The quantitative measurements of lumen characteristics leads to the objective setting of treatment parameters, which in turn results in more reliable and repeatable outcomes, thus diminishing over- and under-treatment risks. Furthermore, procedural time is reduced as treatment parameters are automatically set.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A device for performing surgical procedures, optimally, in body lumens using a local energy source, based on quantitative information, at least partially generated from said device, to determine optimal treatment parameters, comprising:
at least one radiation source;
at least one optical waveguide, having a proximal end and a distal end;
a screen display;
means for measuring body lumen characteristics;
at least one signal processing module;
a calculation module;
means for automatic/manual pullback of said optical fiber;
wherein at said proximal end, said waveguide is optically coupled to said radiation source, and said waveguide transmits said radiation to a treatment site at its distal end;
a treatment device coupled to said at least one waveguide; and
wherein, during operation, said radiation source produces radiation at a wavelength, a power level, a wave pattern, and a power density determined by said calculation module's use of data, accumulated from said means to measure body lumen characteristics, to calculate optimal radiation output from said radiation source.

2. The device for performing procedures in body lumens according to claim 1, wherein said lumen characteristics comprise lumen diameter, tissue reflectivity and presence of ramifications.

3. The device for performing procedures in body lumens according to claim 1, wherein said means for measuring lumen characteristics is selected from the group consisting of a Radiation Emitter/Receptor system, an Ultrasound transducer, Magnetic Resonance imaging, Computerized Axial Tomography imaging, and Optical Coherence Tomography imaging.

4. The device for performing procedures in body lumens according to claim 1, further comprising signaling means to warn physician that radiation parameters are beyond preconfigured limits.

5. The device for performing procedures in body lumens according to claim 1, wherein said calculation module determines optimal pullback speed.

6. The device for performing procedures in body lumens according to claim 1, wherein said calculation module determines optimal radiation parameters.

7. The device for performing procedures in body lumens according to claim 1, wherein said calculation is based on a physical and mathematical model of said body lumen.

8. The device for performing procedures in body lumens according to claim 1, wherein said pullback system comprises a visual/audible signal for guiding physician regarding optimal pullback speed.

9. The device for performing procedures in body lumens according to claim 1, further comprising means for storing said lumen characteristics as said optical waveguide is moved within said body lumen.

10. The device for performing procedures in body lumens according to claim 1, further comprising means for storing and retrieving said stored radiation parameters and said pullback speed according to said measured lumen characteristics.

11. A method for performing procedures in body lumens using a local energy source, based on quantitative information for determining optimal treatment parameters, comprising the steps of:
a) Preparing patient;
b) Inserting radiation conveying means;
c) Measuring lumen characteristics;
d) Calculating optimal radiation parameters;
e) Calculating optimal pullback speed; and
f) Applying radiation to lumen.
